(19) United States
(12) Patent Application Publication
(10) Pub. No.: US 2024/0253000 A1
(43) Pub. Date: Aug. 1, 2024

(54) MIXING DEVICE AND A SINGLE-USE DEVICE FOR A MIXING DEVICE

(71) Applicant: Levitronix GmbH, Zürich (CH)

(72) Inventors: Thomas HOLENSTEIN, Umiken (CH); Nils SCHLEGEL, Untersiggenthal (CH); Florian WASSMER, Aarau (CH)

(21) Appl. No.: 18/416,692

(22) Filed: Jan. 18, 2024

(30) Foreign Application Priority Data
Jan. 31, 2023 (EP) 23154333.1

(51) Int. Cl. B01F 35/32; B01F 27/808; B01F 27/90; B01F 33/453; B01F 35/511; B01F 35/513; B01F 101/22; B01F 101/44; C12M 1/00; C12M 1/06

(52) U.S. Cl. CPC B01F 35/3213; B01F 27/808; B01F 27/90; B01F 33/453; B01F 35/511; B01F 35/513; C12M 23/28; C12M 27/02; B01F 2101/22; B01F 2101/44; B01F 2215/0422

(57) ABSTRACT

A mixing device for mixing at least two substances includes a single-use device and a reusable device. The single-use device includes an impeller, vanes, and a cup for receiving the rotor housing. A magnetically effective core is in the rotor housing. The reusable device includes a stator to contactlessly magnetically drive the impeller about the axis of rotation and magnetically levitate the impeller with respect to the stator. A plurality of stabilizing elements is on the impeller for stabilizing the impeller against tilting. Each stabilizing element is arranged between two vanes and extends from a radial inner edge to a radial outer edge with respect to the radial direction, and each radial outer edge has a distance from the axis of rotation which is smaller than half the outer diameter of the impeller.

### CROSS-REFERENCE TO RELATED APPLICATION

This application claims priority to European application Ser. No. 23/154,333.1, filed Jan. 31, 2023, the contents of which are hereby incorporated by reference.

### BACKGROUND

### Technical Field

The disclosure relates to a mixing device for mixing at least two substances and to a single-use device for a mixing device.

### Background Information

Conventional mixing devices for mixing at least two substances, for example two liquids or a liquid with a powder or liquids or suspensions with gases, are used in many technical fields. In many applications, the purity of the mixing container in which the mixing takes place and of the components contained therein is of very great importance. The pharmaceutical industry and the biotechnological industry can be named as examples here. Solutions and suspensions are frequently prepared here, which require a careful blending of the substances.

### SUMMARY

In the pharmaceutical industry, for example in the production of pharmaceutically active substances, very high demands are made on purity, the components which come into contact with the substances often even have to be sterile. Similar requirements also result in biotechnology, for example in the preparation, treatment or cultivation of biological substances, cells or microorganisms, where an extremely high degree of purity has to be ensured in order not to endanger the usability of the product produced.

For example, bioreactors are used for the recovery of substances, for example proteins, or for the cultivation of cells or other biological material. Bioreactors can be operated both in continuous processes and in batch processes. In these processes, it is a typical method to remove the fluid, for example a cell broth, from the bioreactor, feed it to a filter device, and return the retentate to the bioreactor again. The substance to be extracted is then removed as filtrate or permeate from the filter device and discharged.

Therefore, mixing devices are required in bioreactors, for example to ensure a continuous mixing of the cell broth or its continuous circulation in the mixing container. Here, a very high degree of purity must be ensured in order to protect the substances, or the products produced from contamination.

Therefore, sterility is of very great importance in such processes where, for example, biological activities take place. Sterilization of the devices, for example by steam sterilization, is very often a time-consuming and cost-intensive factor. For this reason, there is an increasing tendency today to design components of the respective device as single-use parts for such processes to avoid or reduce to a minimum time-consuming sterilization processes. In particular, those components that come into direct contact with the biological substances during the process are often designed as single-use parts. The term single-use parts (single use) refers to parts or components that may only be used once in accordance with their intended purpose. After use, the single-use parts are disposed of and replaced for the next application by new, i.e., not yet used, single-use parts.

The single-use parts are sterilized before use, for example by being applied with gamma radiation. These single-use parts or single-use devices must then be assembled with other components, for example, a reusable device designed for multiple use. When manufacturing or designing single-use devices for single use, it is an important criterion that they can be assembled with the reusable device or its components in the simplest possible way. It is desirable that this assembly can be done with as little effort as possible, in a few simple steps, quickly and preferably without tools. Therefore, efforts are being made to design the single-use devices and the reusable devices in such a way that they can be assembled or separated in the simplest possible way.

In order to meet the purity requirements for the process as well as possible, efforts are made to keep the number of components of a mixing device which come into contact with the respective substances as small as possible. For this purpose, electromagnetically operated mixing devices are known in which a rotor, which usually comprises or drives an impeller, is arranged in the mixing container. Then, a stator is disposed outside the mixing container, which drives the rotor through the wall of the mixing container by magnetic or electromagnetic fields without contact and magnetically levitates it in a desired position without contact. This “contactless” concept particularly also has the advantage that no mechanical bearings or feedthroughs into the mixing container are required, which can be a cause of impurities or contamination.

A particularly efficient device of this type, with which substances are circulated or mixed in a bioreactor, is disclosed in the scope of EP-B-2 065 085. Here, the stator and the rotor arranged in the mixing container form a bearingless motor. The term bearingless motor means an electromagnetic rotary drive in which the rotor is levitated completely magnetically with respect to the stator, wherein no separate magnetic bearings are provided. For this purpose, the stator is designed as a bearing and drive stator, which is both the stator of the electric drive and the stator of the magnetic levitation. A magnetic rotating field can be generated with the windings of the stator, which on the one hand exerts a torque on the rotor, which effects its rotation and which, on the other hand, exerts a shear force, which can be set as desired, onto the rotor so that its radial position can be actively controlled or regulated.

In mixing devices with a magnetically levitated rotor or magnetically levitated impeller, problems can arise from the fact that the magnetic levitation is not arbitrarily high loadable. This is especially true for designs in which at least one degree of freedom of the rotor is only passively magnetically stabilized by reluctance forces, i.e., it cannot be actively controlled or regulated. If the forces or moments on the impeller affecting this degree of freedom become too great, a reliable magnetic levitation of the rotor is no longer guaranteed. As an example, tilting of the rotor or the impeller with respect to the axial direction defined by the desired axis of rotation can be mentioned here. If the tilting moments acting on the impeller in the operating state become too great, the reluctance forces stabilizing the rotor are no longer sufficient to generate sufficiently large restoring torques, so that the impeller can, for example, strike the wall(s) surrounding the impeller.

Starting from this state of the art, it is therefore an object of the disclosure to propose a mixing device with a magnetically levitated impeller, which comprises a single-use device for single use and a reusable device for multiple use, wherein the impeller is better secured against excessive tilting. Furthermore, it is an object of the disclosure to propose a single-use device for such a mixing device.

The subject matters of the disclosure meeting these object are characterized by the features described herein.

According to the disclosure, a mixing device for mixing at least two substances is proposed, having a single-use device designed for single use and a reusable device designed for multiple use, wherein the single-use device comprises an impeller having a rotor housing and a plurality of vanes for mixing the substances, and a cup for receiving the rotor housing, wherein the impeller is designed for rotating about a desired axis of rotation defining an axial direction, wherein the impeller has an outer diameter which is the maximum extension of the impeller in a radial direction perpendicular to the axial direction, wherein a magnetically effective core is provided in the rotor housing, wherein the cup is designed in a dimensionally stable manner, wherein the reusable device comprises a stator, by which, in the operating state, the impeller can be contactlessly magnetically driven for rotation about the desired axis of rotation and can be magnetically levitated with respect to the stator, wherein the stator comprises a plurality of windings for generating an electromagnetic rotating field for driving and levitating the impeller, and wherein the stator has a stator housing with a recess which is designed for receiving the cup with the rotor housing arranged therein, so that the cup can be inserted into the recess. A plurality of stabilizing elements is disposed on the impeller for stabilizing the impeller against tilting, wherein each stabilizing element is arranged between two vanes and extends from a radial inner edge to a radial outer edge with respect to the radial direction, and wherein each radial outer edge has a distance from the desired axis of rotation which is smaller than half the outer diameter of the impeller.

It has been shown that a significantly better stabilization of the rotating impeller against tilting relative to the axial direction results from these stabilizing elements on the impeller, which are arranged on the vanes and/or at the rotor housing. Furthermore, the axial position of the impeller is also better stabilized, so that the impeller remains better in its desired position with respect to the axial direction and displaces less easily in the axial direction. If the mixing device is designed according to the principle of the bearingless motor, the impeller is usually passively magnetically stabilized or levitated, i.e., it cannot be controlled, with respect to these three degrees of freedom, namely the two degrees of freedom of tilting and the degree of freedom of the axial position. The magnetic levitation is then relieved by the stabilizing elements precisely with respect to the degrees of freedom that are passively magnetically stabilized. This results in a significantly more stable magnetic levitation of the impeller. Due to the increased stability, the impeller can then also be subjected to greater mechanical loads, for example longer or wider vanes can be used, which has a positive effect on the efficiency of the mixing device.

In particular, it has been shown that the additional stabilization by the stabilizing elements by far exceeds any potential destabilization that may result from turbulence generated by the stabilizing elements.

Embodiments are possible in which each radial outer edge has a distance from the desired axis of rotation that is at most 90% or at most 80% or at most 70% of half the outer diameter of the impeller.

Preferably, each vane is arranged with a radial distance from the desired axis of rotation with respect to the radial direction, wherein the radial distance is different from zero, and wherein the radial inner edge of each stabilizing element has a distance from the desired axis of rotation which is greater than the radial distance. This means that the vanes extend closer to the desired axis of rotation than the radial inner edges of the stabilizing elements with respect to the radial direction, or in other words, with respect to the radial direction, the radial inner edges of the stabilizing elements are further out than the inner beginning of the vanes, i.e., the stabilizing elements begin further out than the vanes.

According to a preferred embodiment, each vane has a front side, a back side, an upper edge and a lower edge, wherein the front side precedes the back side when the impeller rotates, wherein the lower edge and the upper edge delimit the vane with respect to the axial direction, wherein the upper edge is arranged at the end of the vane facing away from the rotor housing, and wherein the stabilizing elements are arranged on the front sides and/or on the back sides of the vanes.

In this embodiment, the stabilizing elements are thus arranged at the front sides or at the back sides of the vanes and thus on the surfaces of the vanes which primarily provide for the mixing of the substances. Since each stabilizing element is arranged on the front side or on the back side of a vane, the stabilizing element is arranged between this vane and its circumferentially adjacent vane, as seen in the circumferential direction.

Preferably, the stabilizing elements are each arranged at least substantially at right angles to the respective front side or back side of the vane, so that the stabilizing elements each extend from the frontside or from the back side in the circumferential direction and enclose an angle of at least approximately 90° with the front side or with the back side. However, it is also possible that the stabilizing elements are arranged obliquely to the front side or to the back side, i.e., they enclose an obtuse angle or an acute angle with the front side or with the back side.

Those embodiments are thus preferred in which the stabilizing elements each enclose an angle different from 0° and from 180° with the front side or with the back side, which is preferably in the range of 40° to 140°.

It is a further advantageous measure that the stabilizing elements are arranged symmetrically, such that each vane, on the front side of which a stabilizing element is provided, is also provided with a stabilizing element on the back side. This symmetrical embodiment with respect to the respective vane results in a balanced axial position of the impeller. In particular, larger deflections of the impeller, for example due to additional tilting moments, can be avoided.

Furthermore, it has proved advantageous if the stabilizing elements are arranged closer to the lower edge than to the upper edge of the respective vane. Then, the stabilizing elements are arranged closer to the magnetically effective core with respect to the axial direction, which has a positive effect on the stabilization of the impeller. It has also proved advantageous with regard to the stabilization achieved to arrange the stabilizing elements closer to the desired axis of rotation with respect to their radial position, so that the respective radial inner edge of the stabilizing element is arranged closer to the radially inner end of the vane than to the radially outer end of the vane.

In summary, it is thus advantageous if the stabilizing elements are arranged close to the center of the magnetically effective core with respect to both the axial direction and the radial direction.

According to a preferred embodiment, each vane is designed in a curved manner so that the front side or the back side are designed in a concave manner.

It is a further preferred embodiment that the stabilizing elements comprise ring segments which each extend from the front side of one vane to the back side of an adjacent vane. In this embodiment, the front side of one vane is connected to the back side of the adjacent vane by a ring segment in each case.

According to a preferred embodiment, the rotor housing is designed cylindrically with an outer housing diameter, and with an upper end face and with a lower end face, which delimit the rotor housing with respect to the axial direction, wherein optionally a hub disk is arranged on the upper end face of the rotor housing, on which the vanes are provided. Thus, it is possible to design the impeller with a hub disk on which the vanes are arranged and then to connect this hub disk to the rotor housing, for example by a snap-lock connection, a bayonet connection, or a snap-on connection. On the other hand, it is also possible that the vanes are arranged directly on or in one of the end faces of the rotor housing.

According to a preferred embodiment, the stabilizing elements are arranged on the upper end face of the rotor housing or on the hub disk, wherein the distance of each radial outer edge from the desired axis of rotation is greater than half the housing diameter of the rotor housing. As a result, the stabilizing elements project beyond the rotor housing with respect to the radial direction.

To increase the clearance available for tilting, it is a preferred measure that the upper end face and/or the lower end face of the rotor housing are designed with a chamfer which extends along the entire circumference of the end face.

It is a further advantageous measure that at least one relief bore is provided which extends completely through the impeller in the axial direction.

Furthermore, a single-use device for a mixing device is proposed by the disclosure, which is designed according to the disclosure. The single-use device comprises the impeller and the cup for receiving the rotor housing.

According to a preferred embodiment, the single-use device further comprises a fixing means or device by which the magnetically effective core is magnetically retained in the cup, wherein the fixing means can be removed by pulling. In this way, the impeller can be fixed in the cup by magnetic forces, for example, when the single-use device is delivered or shipped. After inserting the single-use device into the reusable device and before use, the fixing means is then removed by pulling. The fixing means comprises, for example, a metallic disk which is arranged externally at the bottom of the cup and cooperates with the magnetically effective core of the impeller, and a strap attached to this disk so that the disk can be pulled off the cup.

According to another preferred embodiment, the single-use device further comprises a flexible mixing container for the substances to be mixed, wherein the mixing container is sealingly connected to the cup to form an entirety, and wherein the impeller is arranged in the entirety. The mixing container is designed, for example, as a plastic bag which can be welded to the cup so that the cup with the plastic bag then forms the entirety into which the substances to be mixed are introduced. The flexible mixing container with the cup welded to it can then be inserted into a dimensionally stable support container, wherein the stator then receives the cup with the rotor housing arranged therein.

Further advantageous measures and embodiments of the disclosure are apparent from the dependent claims.

### BRIEF DESCRIPTION OF THE DRAWINGS

In the following, embodiments of the invention will be explained in more detail with reference to the drawings

FIG. 1 illustrates a perspective view of an embodiment of a mixing device according to the disclosure,

FIG. 2 illustrates as FIG. 1, but in an exploded view,

FIG. 3 illustrates a sectional view of the embodiment in a section along the axial direction,

FIG. 4 illustrates as FIG. 3, but in an exploded view,

FIG. 5 illustrates a perspective view of an embodiment of the stator of a mixing device according to the disclosure (without stator housing),

FIG. 6 illustrates a schematic sectional view of the stator from FIG. 5 in a section along the axial direction,

FIG. 7 illustrates a schematic sectional view of an embodiment of the impeller in a section along the axial direction,

FIG. 8 illustrates a perspective sectional view of an embodiment of the rotor housing with a hub disk mounted thereon,

FIG. 9 illustrates a perspective view of an embodiment of a single-use device with a fixing means,

FIG. 10 illustrates the single-use device from FIG. 9 in a section along the axial direction,

FIGS. 11-16 illustrate different variants for the design of the impeller, each in a perspective view,

FIG. 17 illustrates the variant from FIG. 16 in a side view,

FIGS. 18-23 illustrate further variants for the design of the impeller, each in a perspective view, and

FIG. 24 illustrates the variant from FIG. 23 in a plan view.

### DETAILED DESCRIPTION

FIG. 1 shows in a perspective view an embodiment of a mixing device according to the disclosure, which is designated in its entirety by the reference sign 1. Such mixing devices 1 can be used in particular in the biotechnological industry or in the pharmaceutical industry. The mixing device 1 according to the disclosure is also particularly suitable for such applications where a very high degree of purity or sterility of those components coming into contact with the substances to be mixed is essential. The mixing device 1 according to the disclosure can also be designed as a bioreactor or fermenter or as a component of a bioreactor or fermenter. However, it is understood that the disclosure is not limited to such embodiments but relates more generally to mixing devices by which media or substances are mixed. In particular, these substances can be fluids or solids, preferably powders. The mixing device 1 according to the disclosure is suitable for mixing liquids with each other and/or for mixing at least one liquid with a powder or other solids and/or for mixing gases with liquids and/or solids.

In order to guarantee the purity or sterility of those components which come into contact with the substances to be mixed, the mixing device 1 according to the disclosure comprises a single-use device, which is designated in its entirety by the reference sign 100 and which is designed for single use, as well as a reusable device, which is designated in its entirety by the reference sign 200 and which is designed for permanent use, i.e. for multiple use. The single-use device 100 preferably comprises those components which come into contact with the substances to be mixed during the mixing process.

The term “single-use device” and other compositions with the component “single-use”, such as single-use part, single-use component, etc., refer to those devices, components or parts which are designed for single-use, i.e., which can be used only once as intended and are then disposed of. For a new application, a new, previously unused single-use part must then be used. When configuring or designing the single-use device 100, substantial aspects are therefore that the single-use device 100 can be produced as simply and economically as possible, generate few costs and can be produced from materials that are available at the lowest possible price. It is another substantial aspect that the single-use device 100 can be assembled as easily as possible with the reusable device. The single-use device 100 should therefore be able to be replaced very easily without the need for high assembly effort. Particularly preferably, the single-use device 100 should be able to be assembled with the reusable device 200 or separated from the reusable device 200 without the use of tools.

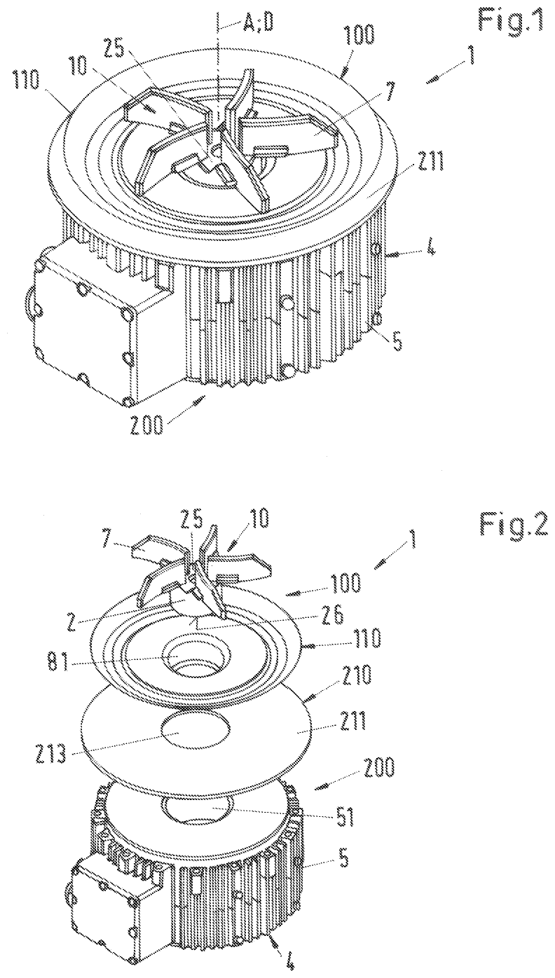

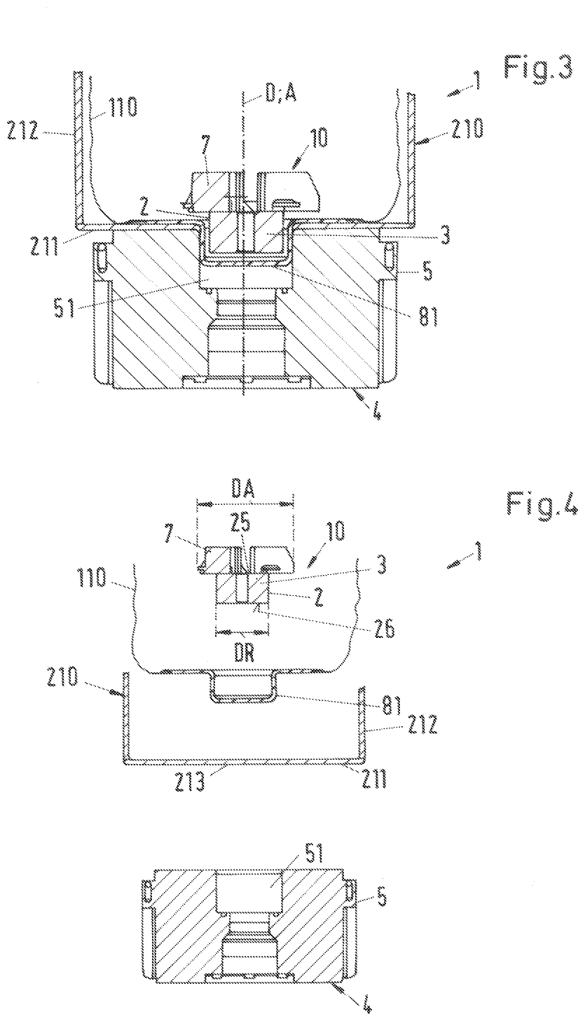

For a better understanding, FIG. 2 still shows a perspective exploded view of the embodiment of the mixing device 1 according to the disclosure from FIG. 1. FIG. 3 shows a sectional view of this embodiment in a section along an axial direction and in the assembled state. Finally, FIG. 4 shows the embodiment in a sectional view analogous to FIG. 3, but in an exploded view.

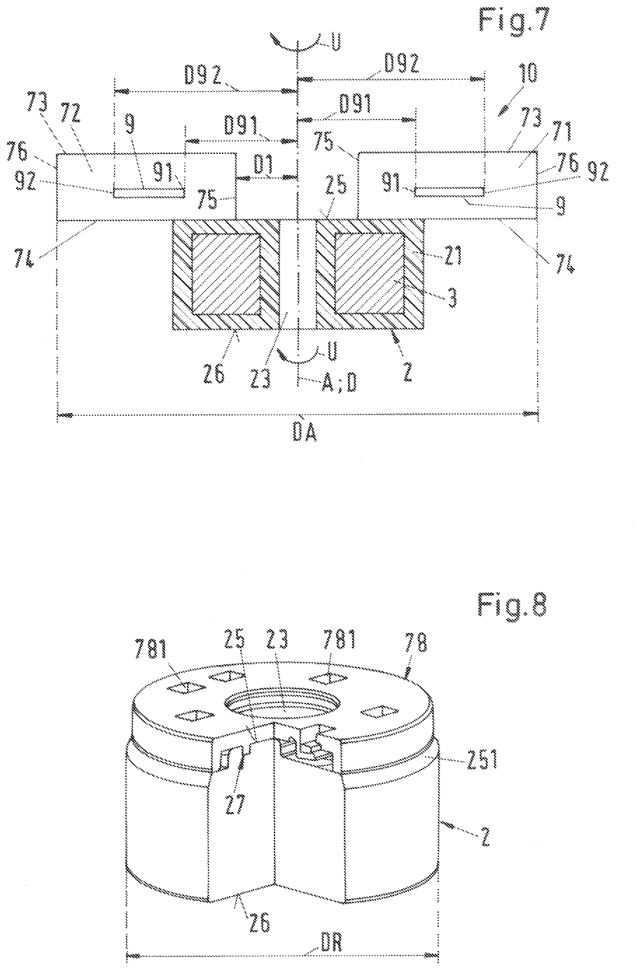

In the mixing device 1 (FIG. 1-FIG. 4), the single-use device 100 comprises at least the following components: an impeller 10 with a rotor housing 2 and with a plurality of vanes 7, in this case with five vanes 7, for mixing the substances, and a cup 81 for receiving the rotor housing 2. A magnetically effective core 3 is arranged in the rotor housing 2, which is designed, for example, in a permanent magnetic manner. The magnetically effective core 3 is fixed with respect to the rotor housing 2 in such a way that the magnetically effective core 3 is both connected in a torque-proof manner to the rotor housing 2 and also cannot perform any translational movement relative to the rotor housing 2. For this purpose, the rotor housing 2 is, for example, cast with a plastic, e.g., with an epoxy resin, whereby the magnetically effective core 3 is fixed relative to the rotor housing 2. It is also possible that the rotor housing 2 is designed as an encapsulation 21 of the magnetically effective core 3 (see e.g., FIG. 7).

The rotor housing 2 is designed cylindrically with an outer housing diameter DR. The rotor housing 2 has an upper end face 25 and a lower end face 26, which delimit the rotor housing 2 with respect to the axial direction A. In the embodiment described here, the five vanes 7 are arranged directly on the upper end face 25 of the rotor housing 2, wherein the vanes 7 are arranged equidistantly on the upper end face 25 with respect to the circumferential direction. For example, the vanes 7 can be placed on the upper end face 25 and attached there, for example by a clamp connection or a snap-lock connection, or the vanes 7 are glued or welded to the upper end face 25. It is also possible that the vanes 7 are an integral part of the rotor housing 2.

According to an also preferred variant (see FIG. 8), a separate hub disk 78 is provided on which the vanes 7 are arranged. This hub disk 78 is then fixed to the upper end face 25 of the rotor housing 2.

The impeller 10 with the rotor housing 2 is designed for rotating about a desired axis of rotation D, which defines the axial direction A, wherein the impeller 10 can be contactlessly magnetically driven and can be magnetically levitated. For this purpose, the magnetically effective core 3 is arranged in the rotor housing 2. The “magnetically effective core 3” of the impeller 10 refers to that area of the impeller 10 which cooperates magnetically with a stator 4 for torque formation and for generating the magnetic levitating forces.

The impeller 10 has an outer diameter DA (FIG. 4) which is the maximum extension of the impeller 10 in a radial direction, wherein the radial direction is perpendicular to the axial direction A.

The cup 81 is designed in a dimensionally stable manner and is preferably made of a plastic. The cup 81 is designed substantially cylindrically and rotationally symmetrical with respect to the axial direction A.

The inner diameter of the cup 81 is dimensioned such that it is slightly larger than the housing diameter DR of the rotor housing 2. Here, slightly is to be understood in the sense that, on the one hand, the rotor housing 2 can rotate freely in the cup 81 without touching the inner wall of the cup 81 and, on the other hand, the distance between the cylindrical outer wall of the rotor housing 2 and the inner wall of the cup 81 is as small as possible in order to ensure an efficient magnetic levitation and an efficient magnetic drive of the impeller 10.

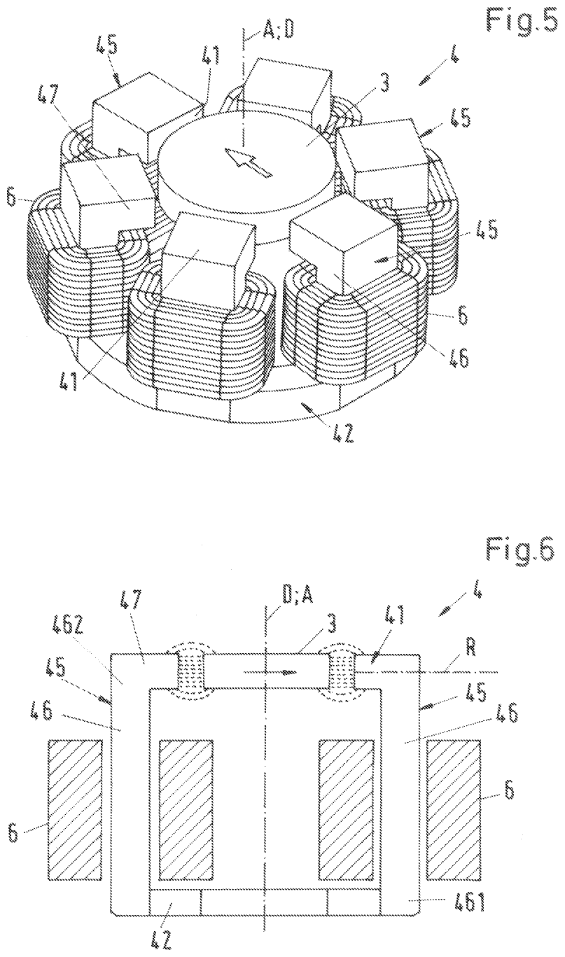

The reusable device 200 comprises the stator 4, with which the impeller in the operating state can be contactlessly magnetically driven for rotation about the desired axis of rotation D and can be magnetically levitated with respect to the stator 4, wherein the stator 4 comprises a plurality of windings 6 (see, for example, FIG. 5) for generating an electromagnetic rotating field for driving and levitating the impeller 10. The stator 4 is arranged in a stator housing 5, which is designed as an encapsulation of the stator 4, preferably as a hermetic encapsulation.

The stator housing 5 is delimited with respect to the axial direction A by two end faces. A central recess 51 is disposed in one of the two end faces, which is designed for receiving the cup 81 with the rotor housing 2 arranged therein, so that the cup 81 can be inserted into the recess 51. Thus, the magnetically effective core 3 arranged in the rotor housing 2 can interact magnetically with the stator 4 and be driven for rotation without contact by the windings 6 of the stator 4. The recess 51 in the stator housing 5 is designed with respect to its radial dimension in such a way that, on the one hand, the cup 81 can be inserted into the recess 51 in a simple manner and that, on the other hand, the distance between the stator housing 5 and the magnetically effective core 3 measured in the radial direction is as small as possible. The dimensions of the cup 81 and the recess 51 are preferably adapted to each other in such a way that the recess 51 closely encloses the cup 81 in the assembled state and abuts with its wall against the shell surface of the cup 81.

Preferably, the single-use device 100 further comprises a flexible mixing container 110 for receiving the substances to be mixed, which is made of a plastic. The flexible mixing container 110 is indicated in FIG. 1 and in FIG. 2 only with its lower edge according to the representation. In FIG. 3 and FIG. 4, somewhat more of the mixing container 110 is represented. The mixing container 110 is preferably a flexible bag, for example a plastic bag, which can be folded so that it takes up as little space as possible during storage. The flexible mixing container 110 is sealingly connected to the cup 81 to form an entirety, in which the mixing of the substances takes place. The flexible mixing container 110 is welded or glued to the cup 81, for example.

The mixing container 110 can have at least one inlet (not represented) in a manner known per se through which the substances to be mixed can be introduced into the mixing container 110. Furthermore, further inlets can be provided, for example for feeding additional substances, for accommodating probes or measuring sensors with which parameters are monitored during the mixing process, e.g., temperature, pressure, concentrations, etc. The inlets can also be used for mass transfer, in particular during the mixing process. In particular, necessary gases can be fed or discharged here, for example in an embodiment as a bioreactor. In particular in the cultivation of microorganisms or biological tissue or cells, it is often a necessity that oxygen or air can be fed to the mixing container 110 and other gases, in particular carbon dioxide, can be discharged from the mixing container 110. Furthermore, the mixing container 110 comprises at least one outlet (not represented) through which substances, for example the mixed substances, can be discharged.

Since the flexible mixing container 110 is often adapted to the specific application, for example with respect to the number and arrangement of the inlets, it is a preferred variant that the single-use device 100 is packaged and delivered to the user without the flexible mixing container 110. The user then provides the flexible mixing container 110 suitable for the specific application and sealingly connects it to the cup 81 of the single-use device 100 to form the entirety for receiving the substances to be mixed.

For use, the flexible mixing container 110 with the cup 81 attached thereto is inserted into a dimensionally stable support container 210, which is, for example, a component of the reusable device 200. The support container 210 is designed substantially cylindrically with a circular bottom plate 211 and a cylindrical side wall 212. The side wall 212 is indicated in FIG. 3 and in FIG. 4. For reasons of a better overview, only the bottom plate 211 is represented in FIG. 1 and FIG. 2.

The substantially cylindrically designed supporting container 210 is open on its upper side, so that the mixing container 110 can be inserted into the support container 210 without difficulty. The support container 210 is designed, for example, as a tank and is made, for example, of steel, in particular a stainless steel.

A centrally arranged and circularly designed opening 213 is disposed in the bottom plate 211 of the support container 210, which opening 213 is dimensioned such that the cup 81 can be passed through this opening 213 and is supported at its edge by the bottom plate 211.

The stator housing 5 is arranged on the outside of the bottom plate 211 such that the central recess 51 in the stator housing 5 is aligned with the opening 213 in the bottom plate 211, so that the cup 81 can be inserted through the opening 213 in the bottom plate 211 into the recess 51 of the stator housing 5. Preferably, the stator housing 5 is attached to the outside of the bottom plate 211, for example by screws (not represented).

The assembly of the single-use device 100 and the reusable device 200 to form the mixing device 1 is extremely simple, as well as quick and, in particular, can be carried out without tools. For this purpose, the mixing container 110, which is usually folded up for storage, with the cup 81 sealingly attached thereto is placed in the support container 210, and the cup 81 with the impeller 10 arranged therein is inserted through the opening 213 in the bottom plate 211 into the recess 51 of the stator housing 5. Even then, the mixing device 1 is ready for use. After use, the mixing container 110 together with the cup 81 and the impeller 10 is simply pulled out of the support container 210. This particularly simple and trouble-free connection or separation of the single-use device 100 with or from the reusable device 200 thus takes account of a substantial aspect of the embodiment for single use.

Such embodiments are also possible in which the mixing container 110 is designed as a dimensionally stable mixing container 110, so that no support container 210 is required. If the mixing container 110 is designed as a dimensionally stable mixing container 110, it can be designed for single use and made, for example, of a plastic, or the mixing container 110 can be designed for multiple use, i.e., reusable, and made, for example, of a metallic material such as stainless steel.

If the mixing container 110 is designed as a dimensionally stable mixing container 110, it can also be designed in one piece with the cup 81 so that the cup 81 is an integral component of the mixing container. Of course, such embodiments are also possible in which the cup 81 is a separate component that can be sealingly connected to the dimensionally stable mixing container 110.

In the following, a preferred embodiment of the stator 4 of the mixing device 1 according to the disclosure is explained on the basis of FIG. 5 and FIG. 6. FIG. 5 shows this embodiment of the stator 4 in a perspective view. FIG. 6 shows the stator 4 in a sectional view along the axial direction A. For a better understanding, the stator housing 5 is not represented in FIG. 5 and FIG. 6. Furthermore, for a better understanding, the magnetically effective core 3 of the impeller 10 is shown in FIG. 5 and FIG. 6 in each case, which cooperates with the stator 4 as an electromagnetic rotary drive.

This electromagnetic rotary drive, which comprises the stator 4 and the magnetically effective core 3, is designed here as a temple motor.

In a temple motor, the stator 4 comprises a plurality of coil cores 45, here six coil cores 45, each of which comprises a rod-shaped longitudinal limb 46 extending from a first end 461 in the axial direction A to a second end 462, and a transverse limb 47 arranged at the second end 462 of the longitudinal limb 46. Each transverse limb 47 extends in a radial direction toward the magnetically effective core 3 and is delimited by an end face facing the magnetically effective core 3.

The first ends 461 of all longitudinal limbs 46 are magnetically conductively connected to each other via a reflux 42. The reflux 42 is preferably designed in a ring-shaped manner or comprises several segments that connect the longitudinal limbs 46 to one another. Both the reflux 42 and the coil cores 45 of the stator 4 are each made of a soft magnetic material because they serve as flux conducting elements to guide the magnetic flux. Suitable soft magnetic materials for the coil cores 45 and the reflux 42 are, for example, ferromagnetic or ferrimagnetic materials, i.e., in particular iron, nickel-iron, cobalt-iron, silicon iron or Mu-metal. In this case, a design as a stator sheet stack is preferred for the stator 4, in which the coil cores 45 and the reflux 42 are designed in sheet metal, i.e., they consist of several thin sheet metal elements, which are stacked.

Thus, each coil core 45 has a shape of an L, wherein the longitudinal limbs 46 each form the long limb of the L extending in the axial direction A, and the transverse limbs 47 extending perpendicular to the longitudinal limbs 26 in the radial direction toward the magnetically effective core 3 each form the short limb of the L. The plurality of the rod-shaped longitudinal limbs 46, which extend in the axial direction A and are reminiscent of the columns of a temple, have given the temple motor its name.

Each transverse limb 47 forms a stator pole 41 in each case. The stator poles 41 are arranged around the magnetically effective core 3, i.e., the magnetically effective core 3 of the impeller 10 is surrounded by the stator poles 41 in the operating state. The windings 6, which are preferably designed as concentrated windings 6, are arranged at the longitudinal limbs 46. One of the concentrated windings 6 is arranged at each longitudinal limb 46, which surrounds the respective longitudinal limb 46.

In this sense, the term temple motor is to be understood in the context of the present application.

The coil cores 45 are arranged equidistantly on a circular line so that the end faces of the transverse limbs 47 surround the magnetically effective core 3 of the impeller. The magnetically effective core 3 is designed as a permanent magnetic disk or as a permanent magnetic ring which is preferably diametrically magnetized. The magnetization of the magnetically effective core 3 is indicated in each case by the arrow without reference sign in FIG. 5 and in FIG. 6.

The stator 4 of the electromagnetic rotary drive is designed as a bearing and drive stator, with which the impeller 10 can be magnetically levitated without contact with respect to the stator 4 in the operating state.

In addition, the impeller-as already mentioned-can be magnetically driven without contact for rotation about the desired axis of rotation by the stator 4. The desired axis of rotation D refers to that axis about which the magnetically effective core 3 rotates in the operating state when the magnetically effective core 3 is in a centered and not tilted position with respect to the stator 4. This desired axis of rotation D defines the axial direction A. Usually, the desired axis of rotation D defining the axial direction A corresponds to the central axis of the stator 4.

Preferably, the electromagnetic rotary drive 1 is designed according to the principle of the bearingless motor and is operated according to this principle. The term “bearingless motor” means an electromagnetic rotary drive in which the magnetically effective core 3—and thus the impeller 10—is levitated completely magnetically with respect to the stator 4, wherein no separate magnetic bearings are provided. For this purpose, the stator 4 is designed as a bearing and drive stator, which is both the stator 4 of the electric drive and the stator of the magnetic levitation. A magnetic rotating field can be generated with the electrical windings 6 of the stator 4, which on the one hand exerts a torque on the impeller 10, which effects its rotation about the desired axis of rotation D, and which, on the other hand, exerts a shear force, which can be adjusted as desired, on the magnetically effective core 3 in a radial plane R (FIG. 6) perpendicular to the axial direction A, so that its radial position can be actively controlled or regulated. The radial plane R defines the x-y-plane of a Cartesian coordinate system whose z-axis runs in the axial direction A.

Thus, three degrees of freedom of the impeller 10 can be actively regulated, namely its rotation and its radial position (two degrees of freedom). With respect to three further degrees of freedom, namely its position in the axial direction A and tilting with respect to the radial plane R perpendicular to the desired axis of rotation D (two degrees of freedom), the impeller 10 is passively magnetically levitated or stabilized by reluctance forces, i.e., it cannot be controlled. The absence of a separate magnetic bearing with a complete magnetic levitation of the magnetically effective core 3 is the property, which gives the bearingless motor its name. In the bearing and drive stator 4, the levitating function cannot be separated from the drive function.

In the case of a bearingless motor, in contrast to classical magnetic bearings, the magnetic levitation and the drive of the motor is realized by electromagnetic rotating fields. Typically, in the bearingless motor, the magnetic drive and levitation function is generated by the superposition of two magnetic rotating fields, which are usually designated as the drive and control fields. These two rotating fields generated by the windings 6 of the stator 4 usually have a pole pair number that differs by one. For example, if the drive field has the pole pair number p, the control field has the pole pair number p+1 or p−1. In this case, tangential forces acting on the magnetically effective core 3 in the radial plane are generated by the drive field, causing a torque, which causes the rotation about the axial direction A. Due to the superposition of the drive field and the control field, it is also possible to generate a shear force on the magnetically effective core 3 in the radial plane which can be adjusted as desired, with which the position of the magnetically effective core 3 in the radial plane can be regulated. Thus, it is not possible to divide the electromagnetic flux generated by the concentrated windings 6 into an (electro-) magnetic flux that only provides for driving the rotation and an (electro-) magnetic flux that only realizes the magnetic levitation.

To generate the drive field and the control field, it is possible on the one hand to use two different winding systems, namely one to generate the drive field and one to generate the control field. The coils for generating the drive field are then usually designated as drive coils and the coils for generating the control field as control coils. The current impressed in these coils is then designated as the drive current or the control current. On the other hand, it is also possible to generate the drive and levitation function with only one single winding system as in the embodiment described here, so that there is thus no distinction between drive coils and control coils. This can be realized in such a way that the values for the drive current and the control current determined in each case by a control device (not represented) are added or superimposed by calculation—i.e., for example, with the aid of software—and the resulting total current is impressed into the respective concentrated winding 6. In this case, of course, it is no longer possible to distinguish between control coils and drive coils.

In the embodiment of the stator 4 described here, exactly one concentrated winding 6 is thus arranged around each longitudinal limb 46. In the operating state, those electromagnetic rotating fields are generated with these concentrated windings 6 with which a torque is effected on the impeller 10 and with which a shear force, which can be adjusted as desired, can be exerted on the magnetically effective core 3 of the impeller 10 in the radial direction, so that the radial position of the magnetically effective core 3, i.e., its position in the radial plane R perpendicular to the axial direction A, can be actively controlled or regulated.

The control device, which comprises, for example, the power electronics for controlling and supplying the windings 6 as well as the signal processing electronics, is preferably arranged in the stator housing 5.

Further details on the structure and design of the bearingless motor can be found, for example, in EP 4 083 431.

The stator housing 5 preferably forms a jacket around the stator 4. The stator 4 is preferably encapsulated by the stator housing 5. Preferably, the encapsulation of the stator 4 is designed as a hermetic encapsulation. Preferably, the stator housing 5 is made of materials that are electrically non-conductive or at least only slightly conductive and yet durable. In addition, the stator housing 5 is made of materials that are solvent-resistant, so that the stator housing 5 can be cleaned with alcohol or other solvents. For example, plastics such as polyetheretherketone (PEEK) or polyoxymethylene copolymer (POM-C) belong to the non-conductive materials. Metal alloys such as Hastelloy or high-alloy titanium alloys (for example, Grade 5) belong to the relatively poorly conductive materials.

Furthermore, it is a preferred measure that the stator housing 5 with the components of the stator 4 arranged therein is preferably cast with a plastic, for example with an epoxy resin, polyurethane or silicone. This has the advantage that the stator 4 remains very dimensionally stable, even if the stator housing 5 is designed with a thin wall.

FIG. 7 shows in a very schematic sectional view an embodiment of the impeller 10 in a section in the axial direction A. The impeller 10 comprises the rotor housing 2, in which the magnetically effective core 3 is fixed, and the vanes 7, which are arranged on the upper end face 25 of the rotor housing 2 and which are connected to the rotor housing 2 in a torque-proof manner.

The rotor housing 2 comprises the encapsulation 21 or is designed as an encapsulation, by which the magnetically effective core 3 is encapsulated. The encapsulation 21, which completely encloses the magnetically effective core 3, is preferably made of a plastic. For. example, it is possible to cast the magnetically effective core 3 in a plastic. It is also possible to design the rotor housing 2 in two parts, namely with a cup-shaped base part and a lid arranged thereon (not represented). The magnetically effective core 3 is then inserted into the base part and fixed there. Subsequently, the lid is placed on the base part and firmly connected to it, for example by welding or bonding. The fixing of the magnetically effective core 3 in the base part can be achieved by a press fit, for example, so that an interference fit is produced. However, it is also possible to cast the base part with a plastic after insertion of the magnetically effective core 3 in order to fix the magnetically effective core 3 with respect to the rotor housing 2.

In the embodiment represented in FIG. 7, a relief bore 23 is provided which extends completely through the rotor housing 2 and thus through the impeller 10 in the axial direction A. The relief bore 23 is arranged centrally in the rotor housing 2 and extends from the center of the upper end face 25 to the center of the lower end face 26. The relief bore 23 is arranged centrally in the rotor housing 2 and extends from the center of the upper end face 25 to the center of the lower end face 26. The relief bore 23 can, for example—as represented in FIG. 7—be designed as a cylindrical bore. However, it is also possible to make the relief bore conical or to provide a plurality of relief bores, which are all parallel to each other, and which are arranged around the center of the upper end face 25 or the lower end face 26, wherein the arrangement is preferably symmetrical with respect to the centers of the end faces 25, 26.

As is known per se, the relief bore(s) 23 serves/serve to compensate at least partially for the axial thrust generated by the rotating vanes 7, thereby relieving the axial levitation of the impeller 10.

In the embodiment represented in FIG. 7, the magnetically effective core 3 is designed in an annular shape and extends around the relief bore 23. The magnetically effective core can be designed as a permanent magnet or comprise one or more permanent magnets. It is also possible to design the magnetically effective core as a reluctance rotor, i.e., without permanent magnets.

Each vane 7 extends in a generally radial direction from a radially inward beginning 75 to a radially outward end 76 which projects beyond the rotor housing 2 with respect to the radial direction. Each vane 7 is arranged with a radial distance D1 from the desired axis of rotation D, i.e., the radially inward beginning 75 of each vane 7 has the radial distance D1 different from zero from the desired axis of rotation D. Preferably, the radial distance D1 is identical for all vanes 7.

Each vane 7 has a front side 71, a back side 72, an upper edge 73 and a lower edge 74.

The front side 71 and the back side 72 are the faces of the vane 7 which are primarily responsible for mixing the substances. The front side 71 precedes the back side 72 when the impeller 10 rotates. The rotation of the impeller is indicated by the arrows with the reference sign U in FIG. 7. Thus, the front side 71 is that side of the vane 7 which is sometimes designated as the pressure side, while the back side 72 is that side of the vane 7 which is sometimes designated as the suction side.

The upper edge 73 and the lower edge 74 delimit the vane 7 with respect to the axial direction A, wherein the upper edge 73 is arranged at that end of the vane 7, which faces away from the rotor housing 2, and wherein the lower edge 74 is that edge, which is arranged on or in the rotor housing 2.

Furthermore, a stabilizing element 9 is provided at each vane 7 for stabilizing the impeller 7 against tilting. The arrangement and design of the stabilizing elements 9 will be discussed in detail later.

Each stabilizing element 9 is arranged on the front side 71 or on the back side 72 of a vane 7. Of course, a stabilizing element 9 can also be arranged on each front side 71 and on each back side 72 in each case (see, for example, FIG. 15).

Each stabilizing element 9 extends in each case with respect to the radial direction from a radial inner edge 91 to a radial outer edge 92 of the stabilizing element 9. Each stabilizing element 9 is designed and arranged in such a way that each radial outer edge 92 has a distance D92 from the desired axis of rotation D which is smaller than half the outer diameter DA of the impeller 10. This means that the stabilizing elements 9 are not arranged at the radially outward end 76 of the respective vane 7 but are spaced from it with respect to the radial direction. With respect to the radial direction, the stabilizing elements 9 thus end further inwards than the vanes 7.

Embodiments are possible in which each radial outer edge 92 has a distance D92 from the desired axis of rotation D which is at most 90% or at most 80% or at most 70% of half the outer diameter AD of the impeller 10.

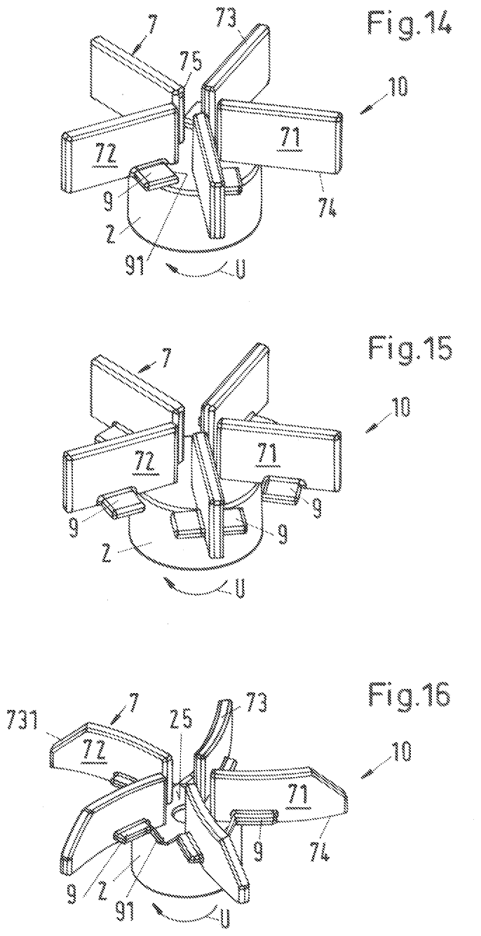

Embodiments are also possible in which each radial outer edge 92 has a distance D92 from the desired axis of rotation D which is at most half, i.e. at most 50%, of half the outer diameter AD of the impeller 10, see e.g. FIG. 14.

Furthermore, it is preferred that the radial inner edge 91 of each stabilizing element 9 has a distance D91 from the desired axis of rotation D which is greater than the radial distance D1 of the vanes 7 from the desired axis of rotation D. This means, with respect to the radial direction, the radial inner edges 91 of the stabilizing elements 9 lie further out than the radially inward beginning 75 of the vanes 7.

FIG. 8 shows in a perspective sectional view an embodiment in which a hub disk 78 is placed on the rotor housing 2. The vanes 7 and the magnetically effective core 3 are not represented in FIG. 8. For a better understanding, a segment is cut out of the rotor housing 2 and the hub disk 78 in the perspective view of FIG. 8.

The magnetically effective core 3 (not represented in FIG. 8) is encapsulated, preferably hermetically encapsulated, in the rotor housing 2. The hub disk 78 is arranged on the upper end face 25 of the rotor housing 2 and has a diameter which is at least approximately equal to the housing diameter DR of the rotor housing 2 or the diameter of the upper end face 25.

As can be recognized in FIG. 8, the upper end face 25 is designed with a chamfer 251 extending along the entire circumference of the end face 25, i.e., the transition area from the upper end face 25 into the shell surface of the rotor housing 2 is designed in a chamfered manner.

Alternatively, or complementarily, the lower end face 26 is designed with a chamfer 261 (not represented in FIG. 8, but see, e.g., FIG. 22) extending along the entire circumference of the lower end face 26.

Particularly preferably, both the upper end face 25 and the lower end face 26 of the rotor housing 2 are each designed with a chamfer 251, 261. The tilting clearance which the impeller 10 has is increased by the chamfers 251, 261. The impeller 10 can be tilted further with the chamfers 251, 261, without the rotor housing 2 coming into physical contact with the cup 81.

The hub disk 78 is fixedly connected to the rotor housing 2. As this is represented in FIG. 8, the hub disk 78 can be connected to the upper end face 25 of the rotor housing 2 via a snap-lock connection 27, so that the hub disk 78 can be snapped onto the upper end face 25. Such snap-lock mechanisms are known to the person skilled in the art in a number of embodiments and do not require further explanation here. Preferably, the snap-lock mechanism is designed in such a way that the forces generating the torque on the impeller 10 do not load the snap-lock mechanism.

Of course, it is also possible to attach the hub disk 78 to the upper face 25 by some other connection, for example by a bayonet connection or a snap-on connection, or with a non-detachable connection such as a weld or a gluing.

Several receptacles 781 are provided in the hub disk 78, into which the vanes 7 can be inserted. The receptacles 781 can be designed in such a way that they simultaneously cause a fixing of the respective vane 7 relative to the hub disk 78. Of course, it is also possible to fix the vanes 7 in the receptacles 781 by other methods, for example by gluing or welding.

Furthermore, embodiments are possible in which the vanes 7 are an integral part of the hub disk 78, i.e., they are designed in one piece with the hub disk 78.

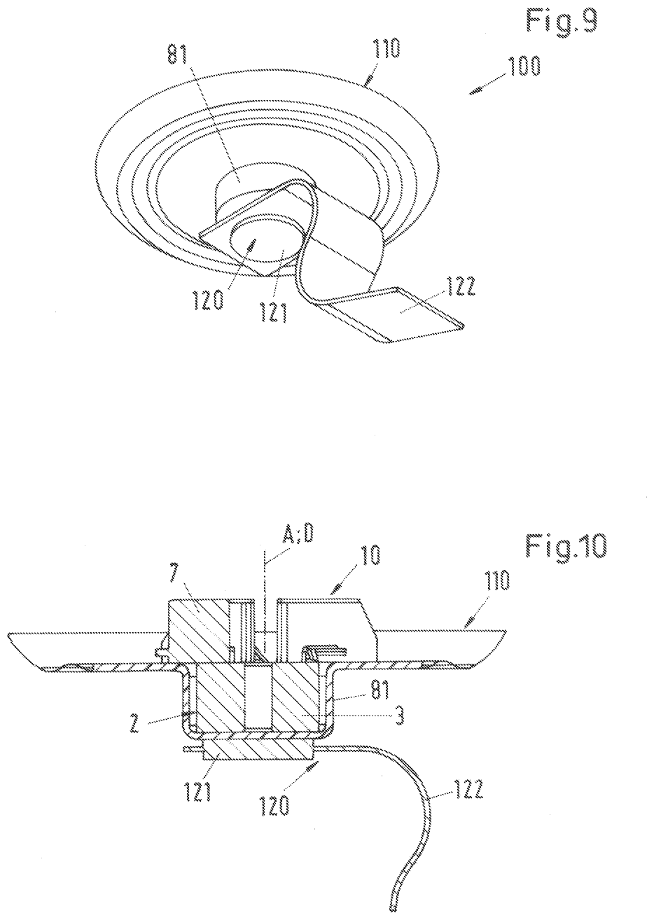

FIG. 9 shows in a perspective view an embodiment of the single-use device 100 with a fixing means or device 120 for the impeller 10 if this is not yet inserted into the stator 4, for example during transport of the single-use device 100. For a better understanding, FIG. 10 still shows this single-use device 100 in a section along the axial direction A.

The single-use device 100 comprises the impeller 10, the cup 81 and the fixing means 120. The fixing means 120 is designed in such a way that it retains the magnetically effective core 3—and thus the impeller 10—in the cup 81 by means of magnetic forces.

For the preferred permanent magnetic embodiment of the magnetically effective core 3, the fixing means 120 comprises a soft magnetic disk 121 to which a strap 122 is attached.

The impeller 10 is inserted into the cup 81 so that the rotor housing 2 with the magnetically effective core 3 arranged therein rests on the bottom of the cup 81. Subsequently, the soft magnetic disk 121 is placed on the bottom of the cup 81 from the outside so that the bottom of the cup 81 is located between the rotor housing 2 and the soft magnetic disk 121. Due to the magnetic interaction between the soft magnetic disk 121 and the permanent magnetic designed magnetically effective core 3, the rotor housing 2—and thus the impeller 10—is fixed in the cup 81.

Before the cup 81 is inserted into the recess 51 of the stator housing 5, the fixing means 120 can be removed by pulling on the strap 122, thereby separating the soft magnetic disk 121 from the bottom of the cup 81.

The fixing means 120 is particularly advantageous for transporting the single-use device 100, because the impeller 10 is held in a well-defined position, namely in the cup 81 by the fixing means 120.

If the magnetically effective core 3 is designed as a reluctance rotor or without a permanent magnet, a permanent magnetic disk is used instead of the soft magnetic disk 121.

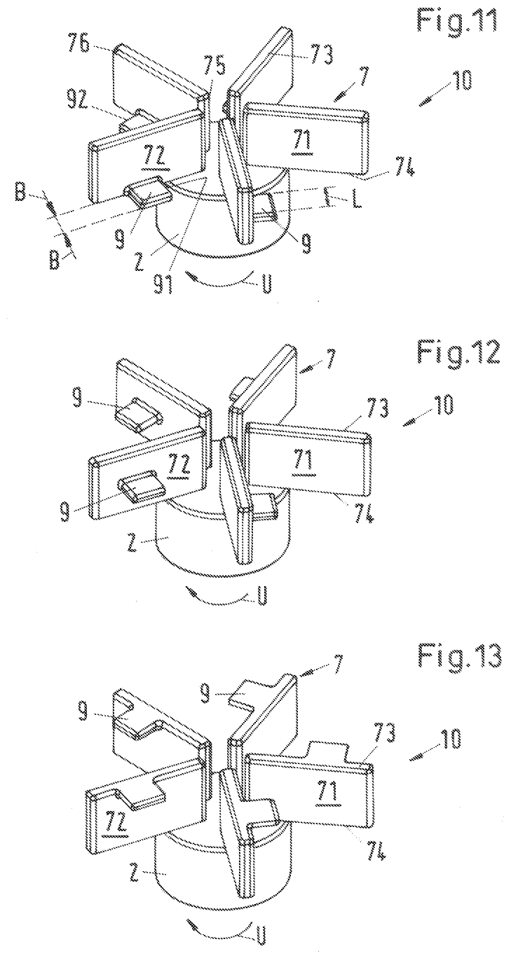

In the following, different variants for the embodiment of the impeller 10 with the stabilizing elements 9 are explained with reference to FIG. 11 to FIG. 24. It is understood that the previous explanations and in particular the explanations referring to FIG. 7 and FIG. 8 also apply in the same way or in analogously the same way to the variants described below.

Furthermore, it should be emphasized that specific measures which are explained by way of example on the basis of one of the variants can also be combined with measures and embodiments which are explained on the basis of other variants, i.e., the measures and embodiments explained in each case can also be combined with other variants.

It is also understood that each of the variants can also be designed with a hub disk.

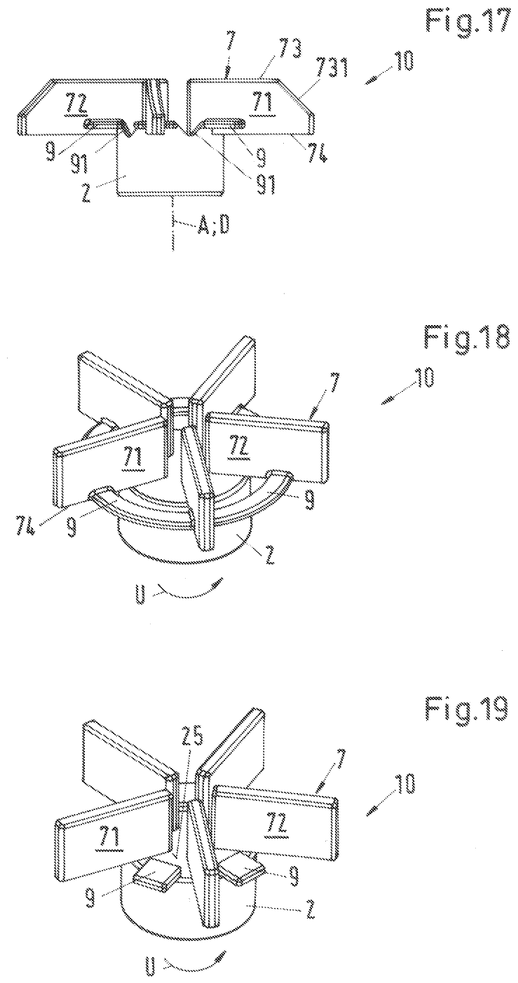

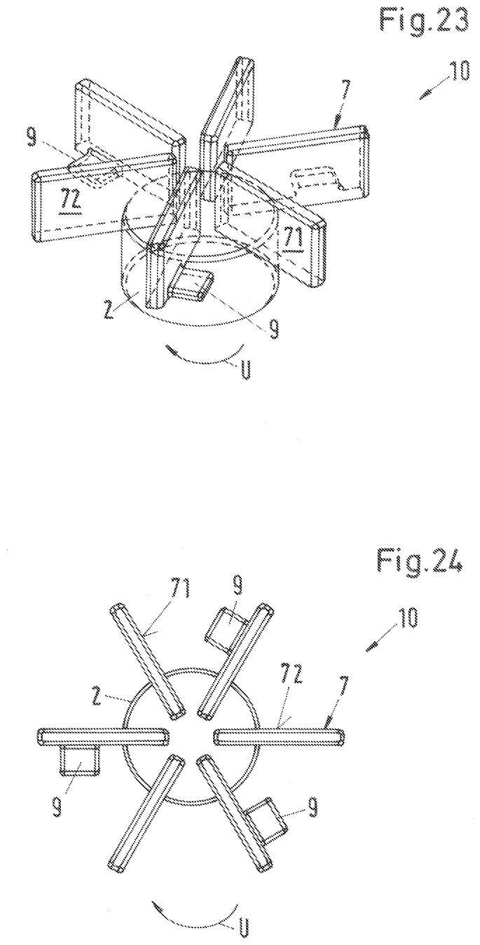

With the exception of FIG. 17 and FIG. 24, all FIGS. 11 to 24 each show a variant for the embodiment of the impeller 10 in a perspective view. FIG. 17 shows the variant from FIG. 16 in a side view. FIG. 24 shows the variant from FIG. 23 in a plan view from the axial direction.

In all variants described in the following, the impeller 10 is designed in each case with exactly five vanes 7, which are arranged equidistantly along the circumference of the impeller 10. Although the embodiment with exactly five vanes 7 is a preferred embodiment, the disclosure is of course not limited to a number of exactly five vanes. The impeller 10 can also be designed with fewer than five vanes 7, for example with exactly three or exactly four vanes 7, or with more than five vanes 7, for example with exactly six or exactly seven vanes 7.

According to the disclosure, the stabilizing elements 9 for stabilizing the impeller 10 against tilting are each arranged between two vanes 7. This feature comprises the two basic possibilities that the stabilizing elements 9 are arranged directly at the rotor housing 2 or, if present, at the hub disk 78, and with respect to the circumferential direction between two vanes 7, or that the stabilizing elements 9 are each arranged at the front side 71 or at the back side 72 of a vane 7. This stabilizing element 9 is arranged between two vanes 7 even in the case of the arrangement at the front side 71 or at the back side 72 of a vane 7, namely between the vane 7 at whose front side 71 or back side 72 the stabilizing element 9 is arranged and the adjacent vane 7 as seen in the circumferential direction. Combinations of these two possibilities can also be realized, namely embodiments of the impeller 10 in which stabilizing elements 9 are arranged both directly at the rotor housing 2 or at the hub disk 78 and at the front sides 71 or back sides 72 of the vanes 7.

FIG. 11 shows a variant in which a stabilizing element 9 is provided in each case at the back side 72 of each vane 7. Each stabilizing element 9 is designed as a rectangular plate, which has a length L and a width B. Here, the length L is the extension of the stabilizing element 9 in the radial direction and the width B is the extension in the direction perpendicular to it and to the axial direction A. Thus, the width B indicates how far the stabilizing element 9 extends into the space between two adjacent vanes 7. All stabilizing elements 9 are designed at least substantially the same. The length L of each stabilizing element 9 is significantly smaller than the corresponding length of the vane 7, wherein the length of the vane 7 is the distance of the radially outward end 76 from the radially inward beginning 75 of the vane 7, measured along the front side 71 (or the back side 72). Preferably, the length L of the stabilizing element 9 is at most half or at most one third of the length of the vane 7.

The stabilizing elements 9 are each arranged at the lower edge 74 of the vane 7 with respect to the axial direction A. The stabilizing elements 9 are arranged approximately centrally at the vane 7 with respect to the radial direction, so that the distance of the radial inner edge 91 of the stabilizing element 9 from the radially inward beginning 75 of the vane 7 is approximately the same as the distance of the radial outer edge 92 of the stabilizing element 9 from the radially outward end 76 of the vane 7.

Of course, it is also possible that the stabilizing elements 9 are each arranged at the front side 71 of a vane 7.

In the variant represented in FIG. 11, the plate-shaped stabilizing elements 9 are each arranged at right angles to the back side 72 of the vane 7, i.e., each stabilizing element 9 encloses an angle of 90° with the back side 72 to which it is arranged. In other embodiments, each stabilizing element 9 is arranged obliquely to the back side 72 (or to the front side 71), so that the stabilizing element 9 encloses an obtuse angle or an acute angle with the respective back side 72 (or front side 71). An angle in the range of 40° to 140° is preferred.

FIG. 12 shows a variant in which the stabilizing elements 9 are arranged approximately centrally between the upper edge 73 and the lower edge 74 of the respective vane 7 with respect to the axial direction A.

FIG. 13 shows a variant in which the stabilizing elements 9 are arranged at the upper edge 73 of the respective vane 7 with respect to the axial direction A.

FIG. 14 shows a variant in which the stabilizing elements 9 are again arranged at the lower edge 74 of the respective vane 7 with respect to the axial direction A but are arranged further inward with respect to the radial direction, i.e., closer to the desired axis of rotation D, compared to the variant shown in FIG. 11. Each stabilizing element 9 is arranged in such a way that its radial inner edge 91 is arranged at or near the radially inward beginning 75 of the respective vane 7.

FIG. 15 shows a variant in which the stabilizing elements 9 are arranged symmetrically, in such a way that each vane 7 has a stabilizing element 9 both on its front side 71 and on its back side 72, wherein the stabilizing elements 9 provided at the same vane 7 are arranged symmetrically with respect to the vane 7, i.e., at the same position with respect to the axial direction A as well as with respect to the radial direction.

Of course, all positions with respect to the axial direction A and with respect to the radial direction are possible, in particular the positions represented in FIG. 11-FIG. 14.

The symmetrical arrangement is a particularly preferred measure.

FIG. 16 and FIG. 17 show a variant which has the additional advantage that the mechanical strength of the vane 7 is significantly increased by the stabilizing elements 9. FIG. 16 shows the variant in a perspective view and FIG. 17 shows the variant in a side view. In this variant, the stabilizing elements 9 are arranged with respect to their position in axial direction A in each case near the lower edge 74 of the vane, in any case significantly closer to the lower edge 74 than to the upper edge 73.

The radial inner edges 91 of the stabilizing elements 9 are designed in such a way that they are each drawn up to the upper end face 25 of the rotor housing 2, so that each stabilizing element 9 is physically connected not only to the respective vane 7 but also to the rotor housing 2. The resulting increase in the mechanical strength of the vane 7 allows larger and, in particular, longer vanes 7. As a result, the mixing device 1 can be operated with slower rotating vanes 7 while maintaining the same mixing efficiency, thus shifting the beginning of undesirable cavitations to higher torques.

As a further measure, the vanes 7 are designed in a curved manner as seen in the radial direction, so that the front side 71 of the vanes is designed in a convex manner in each case and the back side 72 is designed in a concave manner in each case.

As a further option, the upper edges 73 of the vanes 7 include a chamfer 731 in their radially outer area, so that the extension of the vane 7 in the axial direction A decreases in this radially outer area as the distance from the desired axis of rotation D increases. The chamfer 731 can be designed in a straight manner or in a curved manner.

Further possibilities for the embodiment are that the lower edge 74 of the vanes 7 is also designed with a chamfer in its radially outer area, or that the upper edge 73 and/or the lower edge 74 each include a straight-line or a curved chamfer in their radially inward area. Embodiments are also possible in which each vane 7 is designed in such a way that it tapers continuously with increasing distance from the desired axis of rotation D, i.e., it becomes narrower with respect to the extension in the axial direction A.

FIG. 18 shows a variant in which the stabilizing elements 9 are designed as ring segments, wherein each stabilizing element 9 extends in each case from the front side 71 of a vane 7 to the back side 72 of the adjacent vane 7 in the circumferential direction, and wherein the stabilizing element is firmly connected to both vanes 7.

In the embodiment shown in FIG. 18, the ring-segment-shaped stabilizing elements 9 are each arranged at or adjacent to the lower edge 74 of the vane 7. Of course, embodiments are also possible in which the ring-segment-shaped stabilizing elements 9 are arranged at other positions with respect to the axial direction A.

In general, those embodiments are preferred in which the stabilizing elements 9 are arranged close to the magnetically effective core 3 with respect to the axial direction A and further inward with respect to the radial direction, i.e., closer to the desired axis of rotation D.

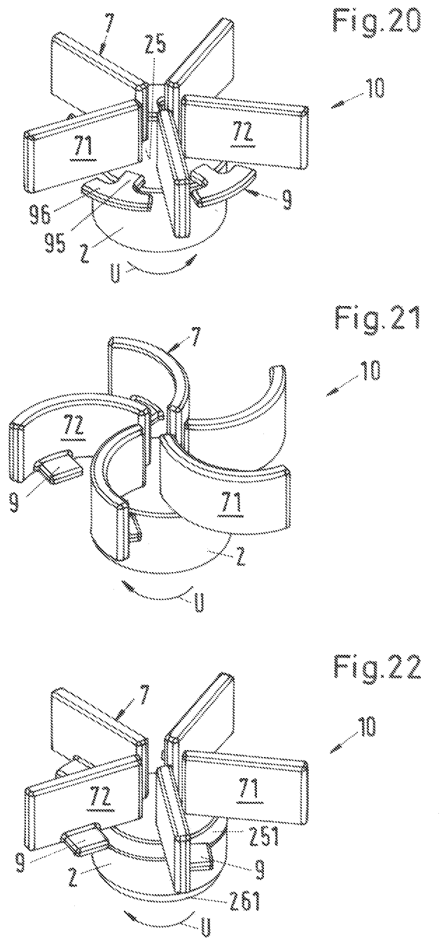

In FIG. 19 and FIG. 20, variants are represented in which the stabilizing elements 9 are arranged directly at the rotor housing 2, namely on the upper end face 25 of the rotor housing 2. It is understood that in embodiments with the hub disk 78, the stabilizing elements 9 are arranged on the hub disk 78 in analogously the same way.

In the variant represented in FIG. 19, a stabilizing element 9 is arranged in each case centrally with respect to the circumferential direction between two adjacent vanes 7 on the upper end face 25 of the rotor housing 2, which projects beyond the rotor housing 2 with respect to the radial direction. Here, the stabilizing elements 9 are thus not directly connected to the vanes 7. The stabilizing elements 9 are designed, for example, as rectangular plates with length L and the width B (FIG. 11).

In the embodiment represented in FIG. 20, the stabilizing elements 9 are each designed in the shape of a T, wherein one end of the longitudinal limb 95 of the T is connected to the upper end face 25 of the rotor housing 2, and the transverse limb 96 of the T is arranged radially outward. In addition, the transverse limb 96 of the T is designed in an arcuate curved manner.

FIG. 21 shows a variant in which the vanes 7 are again designed in a curved manner, so that the front side of the vane 71 is designed in a convex manner and the back side 72 in a concave manner. In this variant, a stabilizing element 9 is provided in each case only on the back side 72 of each vane 7.

FIG. 22 shows a variant in which the rotor housing 2 is designed in each case with a chamfer 251, 261 at both its upper end face 25 and its lower end face 26. This embodiment with the two chamfers 251 and 261 is preferred for all embodiments of the impeller 10.

It is understood that in those embodiments in which the stabilizing elements 9 are arranged directly at the front side 71 and/or at the back side 72 of the vanes 7, a stabilizing element 9 need not be provided on each vane 7.

FIG. 23 and FIG. 24 show such a variant in which the stabilizing elements 9 are arranged directly at the vanes 7, but a stabilizing element 9 is not provided on each vane 7. Thus, variants are also possible in which some of the vanes 7 are designed with a stabilizing element 9 and some vanes 7 are designed without a stabilizing element 9. FIG. 23 shows such a variant in a perspective view, and FIG. 24 shows the variant in a plan view from the axial direction. In this variant, the impeller 10 has exactly six vanes 7. Seen in the circumferential direction, a stabilizing element 9 is arranged in each case at the back side 72 of each second vane 7. Those vanes 7, which are each arranged between two vanes 7 with stabilizing element 9, have no stabilizing elements. As a consequence, three of the vanes 7 have a stabilizing element 9 and the other three vanes 7 have no stabilizing element 9.

Of course, there are many other possible embodiments, for example, the vanes 7—in a straight design—can be arranged in such a way that they do not extend in the radial direction but are arranged inclined against the radial direction. Embodiments are also possible in which the vanes 7 are arranged inclined against the axial direction A in such a way that the lower edge 74 of the vane 7 is arranged offset with respect to the circumferential direction to the upper edge 73 of the same vane 7.

For the mixing device 1 according to the disclosure, which comprises the single-use device 100 and the reusable device 200, it is further an important aspect that the single-use device 100 must be sterilizable for some applications. In this regard, it is particularly advantageous if the single-use device 100 is gamma-sterilizable. In this type of sterilization, the element to be sterilized is applied with gamma radiation. The advantage of gamma sterilization, for example in comparison with steam sterilization, is in particular that sterilization can also take place through the package. For single-use devices in particular, it is a common practice that the parts are placed in the package after they are manufactured and then stored for a period of time before being shipped to the customer. Sterilization then usually takes place shortly before delivery to the customer or only by the customer. In such cases, sterilization takes place through the package, which is not possible with steam sterilization or other processes.

With regard to the single-use device 100, it is generally not necessary for it to be sterilizable more than once. This is a great advantage, particularly in the case of gamma sterilization, because the application of gamma radiation to plastics can lead to degradation, so that multiple gamma sterilization can render the plastic unusable.

Since sterilization under high temperatures and/or under high (steam) pressure can usually be dispensed with for single-use devices 100, less expensive plastics can be used, for example those that cannot withstand high temperatures or that cannot be subjected to multiple high temperature and pressure levels.

Considering all these aspects, it is therefore preferred to use such plastics that can be gamma-sterilized at least once for the manufacture of the single-use device 100. The materials should be gamma-stable for a dose of at least 40 kGy to enable a single gamma sterilization. In addition, no toxic substances should be generated during gamma sterilization. In addition, it is preferred that all materials that come into contact with the substances to be mixed or the intermixed substances meet USP Class VI standards.

For manufacturing the single-use device 100, the following plastics are suitable, for example: polyvinyl chloride (PVC), polypropylene (PP), polyethylene (PE), low density polyethylene (LDPE), ultra-low density polyethylene (ULDPE), high density polyethylene (HDPE), ethylene vinyl acetate (EVA), polyethylene terephthalate (PET), polyvinylidene fluoride (PVDF), acrylonitrile butadiene styrene (ABS), polyurethane (PU),polyacryl, polycarbonate (PC), polysulfones such as polysulfone (PSU), silicones.

What is claimed is:

1. A mixing device for mixing at least two substances, comprising:
a single-use device configured for single use; and
a reusable device configured for multiple use,
the single-use device comprising an impeller having a rotor housing and a plurality of vanes to mix the substances, and a cup to receive the rotor housing, the impeller configured to rotate about a desired axis of rotation defining an axial direction, the impeller having an outer diameter which is a maximum extension of the impeller in a radial direction perpendicular to the axial direction, a magnetically effective core disposed in the rotor housing, the cup configured in a dimensionally stable manner, the reusable device comprising a stator, by which, in the operating state, the impeller is capable of being contactlessly magnetically driven to rotate about the desired axis of rotation and magnetically levitated with respect to the stator, the stator comprising a plurality of windings to generate an electromagnetic rotating field to drive and levitate the impeller, and the stator having a stator housing with a recess configured to receive the cup with the rotor housing arranged therein, so that the cup is capable of being inserted into the recess,
a plurality of stabilizing elements disposed at the impeller to stabilize the impeller against tilting, each stabilizing element of the plurality of stabilizing elements arranged between two vanes of the plurality of vanes and extending from a radial inner edge to a radial outer edge with respect to the radial direction, and each of the radial outer edges having a distance from the desired axis of rotation which is smaller than half the outer diameter of the impeller.

2. The mixing device according to claim 1, wherein each vane of the plurality of vanes is arranged with a radial distance from the desired axis of rotation with respect to the radial direction, the radial distance being different from zero, and the radial inner edge of each stabilizing element of the plurality of stabilizing elements has a distance from the desired axis of rotation which is greater than the radial distance.

3. The mixing device according to claim 1, wherein each vane of the plurality of vanes has a front side, a back side, an upper edge and a lower edge, the front side preceding the back side when the impeller rotates, for each vane of the plurality of vanes the lower edge and the upper edge delimit the vane with respect to the axial direction, the upper edge arranged at an end of the vane facing away from the rotor housing, and for each vane of the plurality of vanes, a stabilizing element of the plurality of stabilizing elements is arranged on the front side or on the back side of the vane.

4. The mixing device according to claim 3, wherein each stabilizing element of the plurality of stabilizing elements enclose an angle different from 0° and from 180° with the front side or with the back side of a respective vane.

5. The mixing device according to claim 3, wherein the plurality of stabilizing elements are arranged symmetrically, such that for each vane of the plurality of vanes, on the front side of which the stabilizing element of the plurality of stabilizing elements is disposed, another stabilizing element of the plurality of stabilizing elements is disposed on the back side.

6. The mixing device according to claim 3, wherein, for a respective vane, the stabilizing element of the plurality of stabilizing elements is arranged closer to the lower edge than to the upper edge.

7. The mixing device according to claim 3, wherein each vane of the plurality of vanes is designed in a curved manner so that the front side or the back side is concaved.

8. The mixing device according to claim 3, wherein the plurality of stabilizing elements comprise ring segments which each ring segment extending from the front side of a respective vane of the plurality of vanes to the back side of an adjacent vane of the plurality of vanes.

9. The mixing device according to claim 1, wherein the rotor housing is cylindrical with an outer housing diameter, and with an upper end face and with a lower end face, which delimit the rotor housing with respect to the axial direction, and on which the plurality of vanes are provided.

10. The mixing device according to claim 9, wherein the plurality of stabilizing elements are arranged on the upper end face of the rotor housing, and a distance of each radial outer edge from the desired axis of rotation is greater than half the housing diameter of the rotor housing.

11. The mixing device according to claim 9, wherein the upper end face or the lower end face of the rotor housing includes a chamfer which extends along an entire circumference of the end face.

12. The mixing device according to claim 1, further comprising at least one relief boreextending completely through the impeller in the axial direction.

13. A single-use device for a mixing device according to claim 1, wherein the single-use device comprises the impeller and the cup to receive the rotor housing.

14. The single-use device according to claim 13, further comprising a fixing device configured to magnetically retain the magnetically effective core in the cup, and the fixing device is configured to be removed by pulling.

15. The single-use device according to claim 13, further comprising a flexible mixing container for the substances to be mixed, the mixing container is sealingly connected to the cup to form an entirety, and the impeller is arranged in the entirety.

16. The mixing device according to claim 3, wherein each stabilizing element of the plurality of stabilizing elements enclose an angle in the range from 40° to 140° with the front side or with the back side of a respective vane.

17. The mixing device according to claim 1, wherein the rotor housing is cylindrical with an outer housing diameter, and with an upper end face and with a lower end face, which delimit the rotor housing with respect to the axial direction, a disk-shaped hub disk is arranged on the upper end face of the rotor housing, on which the plurality of vanes are provided.

18. The mixing device according to claim 17, wherein the plurality of stabilizing elements are arranged on the hub disk, and a distance of each radial outer edge from the desired axis of rotation is greater than half the housing diameter of the rotor housing.
